(19) United States
(12) Patent Application Publication
(10) Pub. No.: US 2025/0288632 A1
(43) Pub. Date: Sep. 18, 2025

(54) Viral Entry Inhibitors Derived From Botanicals

(71) Applicant: Emory University, Atlanta, GA (US)

(72) Inventors: Cassandra Leah Quave, Atlanta, GA (US); Caitlin Risener, Atlanta, GA (US); Sunmin Woo, Atlanta, GA (US)

(21) Appl. No.: 18/861,441

(22) PCT Filed: Apr. 28, 2023
(86) PCT No.: PCT/US2023/020457

§ 371 Date: Oct. 29, 2024

Related U.S. Application Data
(60) Provisional application No. 63/336,471, filed on Apr. 29, 2022.

(51) Int. Cl. A61K 36/28; A61K 36/11; A61P 31/14

(52) U.S. Cl. CPC A61K 36/28; A61K 36/11; A61P 31/14; A61K 2236/331; A61K 2236/333

(57) ABSTRACT

This disclosure relates to methods of treating a coronavirus infection or other microbial infection comprising administering an effective amount of a plant extract, or compound, or combination of compounds derived therefrom to a subject in need thereof. In certain embodiments, the plant extract is obtained by a process of contacting a segment of a plant a with a solvent providing an extract as reported herein. In certain embodiments, this disclosure contemplates pharmaceutical compositions comprising extracts and components contained therein for use in methods of treating viral or other microbial infections.

### CROSS-REFERENCE TO RELATED APPLICATIONS

This application claims the benefit of U.S. Provisional Application No. 63/336,471 filed Apr. 29, 2022. The entirety of this application is hereby incorporated by reference for all purposes.

### BACKGROUND

Some common colds are due to certain coronavirus (CoV) strains associated with mild symptoms. More dangerous human strains include severe acute respiratory syndrome associated coronavirus (SARS-COV-1) and SARS-COV-2 (also referred to as COVID-19). In humans, SARS-COV-2 can be transferred from individuals who have mild symptoms or are asymptomatic and has caused numerous deaths worldwide. Some individuals experience long-term effects, including fatigue, shortness of breath, brain fog, chest pain, headache, and other symptoms. Antiviral treatments are available; however, many are only effective if administered soon after symptoms start. Other treatments have side effects, diminished efficacy due to drug resistance, or exhibit adverse reactions with other medications. Thus, there is a need to identify improved therapies.

### SUMMARY

This disclosure relates to methods of treating a coronavirus infection or other microbial infection comprising administering an effective amount of a plant extract, or compound, or combination of compounds derived therefrom to a subject in need thereof. In certain embodiments, the plant extract is obtained by a process of contacting a segment of a plant a with a solvent providing an extract as reported herein.

In certain embodiments, this disclosure relates to methods of treating a coronavirus infection comprising administering an effective amount of a plant extract, or compound, or combination of compounds derived therefrom to a subject in need thereof wherein the subject is diagnosed with SARS-COV-2, SARS-COV-1, MERS-COV, HCOV-229E, HCoV-OC43, HCoV-NL63, or HCoV-HKU1, paramyxovirus, flavivirus, filovirus, orthomyxovirus, retrovirus, rhabdovirus, herpesvirus, alphavirus, lentivirus, an endemic human coronavirus, epidemic coronavirus, or pandemic coronavirus.

In certain embodiments, the plant extract is from a plant or plant segment of the Solidago, Pteridium, Rubus, Vaccinium, Salix, or Amorpha genus. In certain embodiments, the segment is the flower, root, rhizomes, bark, fruit, leaf, stem, or seed. In certain embodiments, it is contemplated that the extract is obtained using decoction, maceration, a Soxhlet extractor, microwave-assisted extraction, supercritical carbon dioxide extraction, or with assistance of sonication.

In certain embodiments, the plant extract is from Solidago altissima (Extract 1428). In certain embodiments, the segment is the flower. In certain embodiments, the extract is obtained using a solvent selected from water, room temperature water, below room temperature water, boiling water, MeOH and EtOH, and aqueous MeOH, aqueous EtOH. In certain embodiments, the extract comprises a compound such as a quinic acid derivative, coumaroylquinic acid, 3-O-feruloylquinic acid, a glycosidic triterpene derivative, fatty acid, trihydroxy octadecenoic acid, ester, derivative, or combinations thereof.

In certain embodiments, the plant extract is from a plant of the Pteridium genus. In certain embodiments, the plant is Pteridium aquilimim (Extract 1804). In certain embodiments, the plant segment is the rhizomes. In certain embodiments, the solvent is selected from water, room temperature water, below room temperature water, boiling water, MeOH and EtOH, and aqueous MeOH, aqueous EtOH. In certain embodiments, the extract comprises a compound such as a glycosidic phenolic compound, benzoyl hexose, caffeoyl hexose, a glycosidic phenolic compound, procyanidin derivatives, A-type procyanidin dimer and/or prodelphinidin (one double linkage between first and second units), A-type procyanidin trimer (one double linkage between first and second units), quinic acid derivatives such as coumaroylquinic acid, caffeoylquinic acid, cholestane-type sterol, ester, ester derivative, or combinations thereof.

In certain embodiments, this disclosure contemplates pharmaceutical compositions and nutritional supplements comprising plant material, extracts, and components contained therein for use in methods of treating viral or other microbial infections. In certain embodiments, this disclosure relates to pharmaceutical compositions comprising a plant extract, or compound, or mixture of compounds derived thereof as disclosed herein and a pharmaceutically acceptable excipient.

### BRIEF DESCRIPTION OF THE SEVERAL VIEWS OF THE DRAWINGS

FIG. 1 shows concentration-dependent response data (2-128 μg/mL) against SARS-COV-2 variant for three select extracts (1428, 1749, and 1804) tested against the Brazil/Gamm/P.1 variant, South Africa/Beta/B1.351 variant, India/Delta/B1.617.2 variant, and UK/Alpha/B.1.1.7 variant.

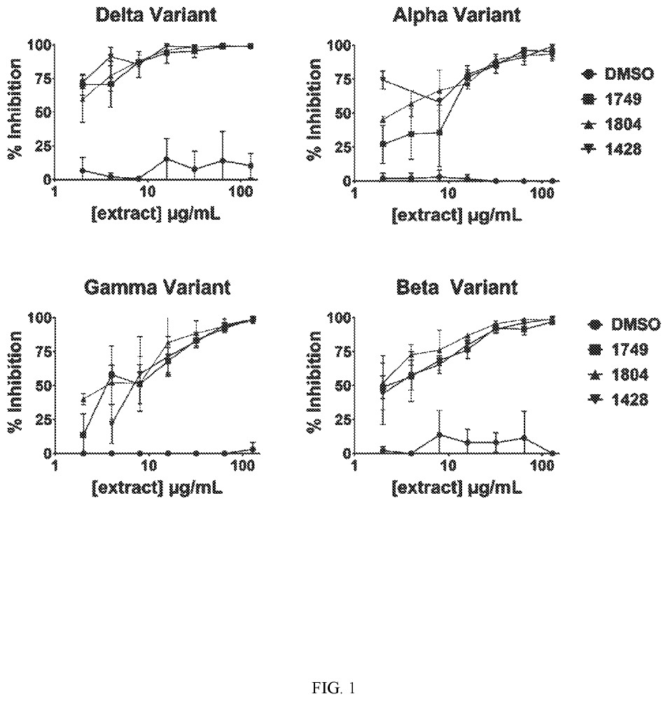

FIG. 2A shows LC-MS base peak ion (BPI) chromatograms of the Pteridium aquilinum 1804 extract. Chromatographic peaks are annotated with peak numbers and associated with compounds in Table 4.

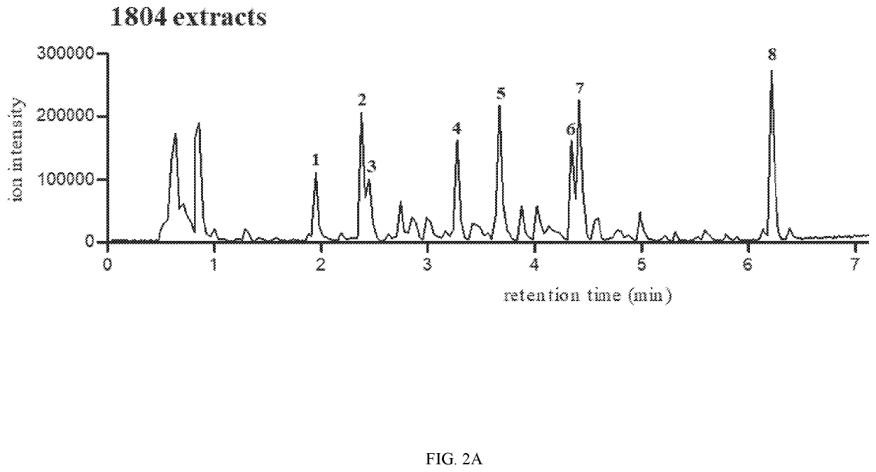

FIG. 2B shows LC-MS base peak ion (BPI) chromatograms of the Solidago altissima 1428 extract. Chromatographic peaks are annotated with peak numbers and associated with compounds in Table 5.

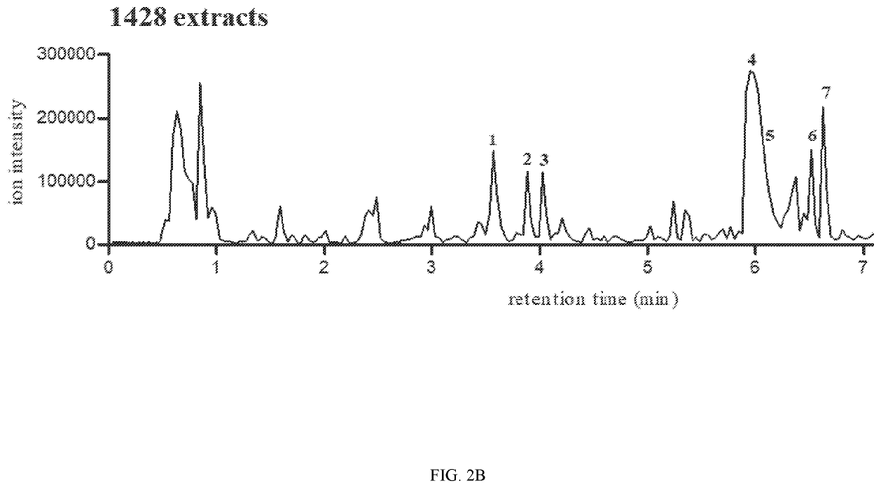

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature. An “embodiment” of this disclosure refers to an example and infers that the example is not necessarily limited to the example.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

“Subject” refers to any animal, preferably a human patient, livestock, rodent, monkey, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

The term “effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing a oxygen atom with a sulfur atom, replacing an amino group with a hydroxyl group, replacing a nitrogen with a protonated carbon (CH) in an aromatic ring, replacing a bridging amino group (—NH—) with an oxy group (—O—), or vice versa. The derivative may be a prodrug. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In typical embodiments, the salts are conventional nontoxic acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)2Ra, —OS(—O)2Ra and —S(—O)2ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in compositions over the parent drug. A prodrug may be converted into the parent drug by various mechanisms, including enzymatic processes and metabolic hydrolysis. Typical prodrugs are esters. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate, and benzoate derivatives of an alcohol, i.e., hydroxy group.

### Extraction and Purification of Compounds by Chromatograph

In certain embodiments, the extracting process comprises the step of mixing a segment from a plant with a polar solvent, such as a liquid comprising methanol, ethanol, ethyl acetate, n-butanol, acetonitrile, acetone, methylene chloride or chloroform, under conditions such that a mixture of compounds in the segment or the plant dissolves in the solvent. In certain embodiments, the process further comprises the step of removing the solvent by evaporation from the mixture of compounds. In certain embodiments, the process further comprises the step of purifying the mixture of compounds by chromatography, e.g., liquid chromatography through a solid absorbent, e.g., wherein the solid absorbent comprises silica gel or alumina. Chromatography refers to the separation of a mixture of compounds dissolved in a fluid called the mobile phase, which carries the compounds through a structure holding another material called the stationary phase. The various compounds or components of the mixture travel at different speeds, causing them to separate. The separation is based on differential partitioning between the mobile and stationary phases. Subtle differences in a partition coefficient of each compound result in differential retention on the stationary phase and thus changing the separation.

In normal-phase chromatography, the stationary phase is polar. In reversed phase, the stationary phase is nonpolar. Typical stationary phases for normal-phase chromatography are silica or organic moieties with cyano and amino functional groups. For reversed phase, alkyl hydrocarbons are the preferred stationary phase. Examples are solid supports containing a surface conjugated with a hydrocarbon chain, e.g., octadecyl (C18), octyl (C8), and butyl (C4).

In normal-phase chromatography, the least polar compounds elute first and the most polar compounds elute last. The mobile phase typically consists of a nonpolar solvent such as hexane or heptane mixed with a slightly more polar solvent such as isopropanol, ethyl acetate, n-butanol, or chloroform. Retention to the stationary phase decreases as the amount of polar solvent in the mobile phase increases. In reversed phase chromatography, the most polar compounds elute first with the most nonpolar compounds eluting last. The mobile phase is generally a binary mixture of water and a miscible polar organic solvent like methanol, acetonitrile, or tetrahydrofuran.

In certain embodiments, methods of extraction comprise mixing a section or part of a plant with an water miscible carbon containing solvent, e.g., such as a protic solvent, an alcohol, methanol, ethanol, 1-propanol, 2-propanol, tetrahydrofuran, acetone, acetic acid, 1,4-dioxane or mixture providing a concentrate with a mixture of compounds and substantially removing the solvent from the concentrate, purifying the solvent derived concentrate to less than 5%, 1%, or 0.5% by weight of the solvent used in the extraction, e.g., evaporating the protic solvent and/or optionally in combination with mixing the concentrate with water, sonicating the water, freezing the water to provide ice, and removing the ice by sublimation (e.g. in a vacuum of low pressure) wherein said purification methods may be repeated in combination. In certain embodiments, the method further comprises suspending the solvent derived concentrate in water and optionally extract impurities in a hydrocarbon solvent such as cyclohexane, heptane, hexane, pentane, 2,2,4-trimethylpentane, separating the hydrocarbon from the water providing a water layer. In certain embodiments, the method further comprises mixing the water layer with a solvent that is immiscible in water (polar and/or aprotic), e.g., such as ethyl acetate, diethyl ether, methyl tertbutyl ether, n-butanol, toluene, methylene chloride, carbon tetrachloride, 1,2-dichloroethant, and/or chloroform, and purifying the solvent to provide a second solvent derived concentrate. In further embodiments, the second derived concentrate is purified one or more times by liquid chromatography, e.g., normal phase chromatography.

In certain embodiments, the disclosure contemplates that liquid chromatography includes using a mobile phase utilizing with a solvent that is immiscible in water (polar and/or aprotic) and increasing amounts of an alcohol. In certain embodiments, the disclosure contemplates that liquid chromatography includes using a mobile phase utilizing dichloromethane and increasing amounts of methanol. In certain embodiments, the disclosure contemplates that liquid chromatography includes using a mobile phase utilizing ethyl acetate and increasing amounts of methanol. In certain embodiments, the disclosure contemplates that liquid chromatography includes using a mobile phase utilizing dichloromethane and increasing amounts of n-butanol. In certain embodiments, the disclosure contemplates that liquid chromatography includes using a mobile phase utilizing ethyl acetate and increasing amounts of n-butanol. In certain embodiments, the disclosure contemplates that liquid chromatography includes using a mobile phase utilizing dichloromethane and increasing amounts of ethanol. In certain embodiments, the disclosure contemplates that liquid chromatography includes using a mobile phase utilizing ethyl acetate and increasing amounts of ethanol. In certain embodiments, the disclosure contemplates that liquid chromatography includes using a mobile phase utilizing dichloromethane and increasing amounts of n-propanol. In certain embodiments, the disclosure contemplates that liquid chromatography includes using a mobile phase utilizing ethyl acetate and increasing amounts of n-propanol.

### Methods of Use

This disclosure relates to methods of treating a coronavirus infection or other microbial infection comprising administering an effective amount of a plant extract, or compound, or combination of compounds derived therefrom to a subject in need thereof, wherein the plant extract is obtained by a process of contacting a segment of a plant a with a solvent providing an extract as reported herein. In certain embodiments, the solvent is selected from water, room temperature water, below room temperature water, boiling water, MeOH and EtOH, and aqueous MeOH, aqueous EtOH. In certain embodiments, it is contemplated that the extract is obtained using decoction, maceration, Soxhlet extractor, microwave-assisted extraction, supercritical carbon dioxide extraction, or with assistance of sonication.

In certain embodiments, this disclosure relates to methods of treating a coronavirus infection or other microbial infection comprising administering an effective amount of a plant extract, or compound, or combination of compounds derived therefrom to a subject in need thereof, wherein the subject is diagnosed with SARS-COV-2, SARS-COV-1, MERS-COV, HCOV-229E, HCoV-OC43, HCoV-NL63, or HCoV-HKU1, paramyxovirus, flavivirus, filovirus, orthomyxovirus, retrovirus, rhabdovirus, herpesvirus, alphavirus, lentivirus, an endemic human coronavirus, epidemic coronavirus, or pandemic coronavirus. In certain embodiments, the extract, compound, or compounds, esters or derivatives are administered with 5 days or after 5 days, or 1 week or 1 month after symptom onset.

In certain embodiments, the subject is more than 55, 65, or 75 years old. In certain embodiments, the subject is diagnosed with a severe acute infection requiring intensive care. In certain embodiments, the subject is any age, e.g., less than 25, 20, 15, or 10 years old or the subject is more than 55, 65, or 75 years old.

In certain embodiments, this disclosure relates to treating or preventing chronic acute respiratory syndrome or associated side effects due to a coronavirus or other viral infections comprising administering a composition comprising an effective amount of an extract or compound disclosed herein to a subject in need thereof. In certain embodiments, the subject is diagnosed with a viral infection that poses a risk of developing an acute respiratory syndrome or chronic acute respiratory syndrome such as subject diagnosed with a high-risk coronavirus infection, e.g., SARS-COV-1 or SARS-COV-2 infection.

In certain embodiments, administration is daily or 2 or more days, 3 or more days, 4 or more days, 5 or more days, 6 or more days, or for a week or more. In certain embodiments, the composition administered daily. In certain embodiments, the composition is administered daily for more than 3, 5, 7 days or two weeks.

In certain embodiments, the compositions are administered daily, up to 2 times a day, 3 times a day, or as a continuous infusion. In certain embodiments, compositions are administered as a continuous enteral feeding for severely ill and/or hospitalized subjects.

In certain embodiments, the subject is diagnosed with fatigue, shortness of breath, anxiety, depression, brain fog, joint pain, and/or chest pain, and optionally diabetes, stroke, heart rhythm abnormality, and/or blood clot in the lungs.

In certain embodiments, the subject is diagnosed with long COVID, e.g., three weeks or a month after an infection the subject has one, two, or more of the following conditions: tiredness or fatigue that interferes with daily life, brain fog, symptoms that get worse after working hard or thinking hard (exertional malaise), fever, respiratory and heart symptoms, difficulty breathing or shortness of breath, cough, chest pain, fast-beating or pounding heart, nervous system symptoms, difficulty thinking or concentrating, headaches, sleep problems, dizziness when standing up lightheadedness, pins-and-needles feelings, change in smell or taste, depression or anxiety, digestive symptoms, diarrhea, stomach pain, joint or muscle pain, rash, and/or changes in menstrual periods.

In certain embodiments, the subject is more than 55, 65, or 75 years old and/or diagnosed with a severe acute infection requiring intensive care, pre-existing respiratory illness, obesity, diabetes, high blood pressure, chronic cardiovascular disease, chronic kidney disease, organ transplant, or cancer.

Although embodiments of this disclosure contemplate treatment of coronavirus infections, management of other viral infections are contemplated such as influenza virus, rhinovirus, hepatitis A, hepatitis B, hepatitis C, human papillomaviruses, human immunodeficiency, herpes virus, Epstein-Barr virus, herpes simplex virus, varicella-zoster virus, shingles virus, mumps virus, measles virus, West Nile virus, poliovirus, non-poliovirus enterovirus, respiratory syncytial virus, and parainfluenza virus.

In certain embodiments, the extract or compound disclosed herein is administered in combination with another anti-viral agent such as, abacavir, acyclovir, adefovir, amantadine, arbidol, baloxavir, boceprevir, daclatasvir, docosanol, edoxudine, enfuvirtide, famciclovir, foscarnet, ganciclovir, ibacitabine, idoxuridine, imiquimod, imunovir, lamivudine, letermovir, marboxil, methisazone, moroxydine, nexavir, oseltamivir, peramivir, penciclovir, pleconaril, ribavirin, rimantadine, simeprevir, sofosbuvir, taribavirin, telbivudine, tenofovir, trifluridine, tromantadine, umifenovir, valaciclovir, vidarabine, zanamivir, zidovudine, or combinations thereof.

In certain embodiments, an extract or compound disclosed herein can be administered in combination with other anti-viral agents such as remdesivir, chloroquine, hydroxychloroquine, azithromycin, ivermectin, lopinavir, ritonavir, nitazoxanide, or combinations thereof.

In certain embodiments, an extract or compound disclosed herein can be administered in combination with other anti-viral agents such as nirmatrelvir and/or ritonavir. In certain embodiments, treatment commences within 5 days or 7 days of when symptoms start.

In certain embodiments, an extract or compound disclosed herein can be administered in combination with remdesivir. In certain embodiments, treatment commences within 5 days or 7 days of when symptoms start.

In certain embodiments, an extract or compound disclosed herein can be administered in combination with molnupiravir. In certain embodiments, treatment commences within 5 days or 7 days of when symptoms start.

In certain embodiments, an extract or compound disclosed herein can be administered in combination with a JAK inhibitor.

In certain embodiments, an extract or compound disclosed herein can be administered in combination with baricitinib.

In certain embodiments, an extract or compound disclosed herein can be administered in combination with convalescent plasma. In certain embodiments, the subject is prone to an overactive immune response, or the subject is immunocompromised or are receiving immunosuppressive treatment.

In certain embodiments, it is contemplated that the subject may have a viral, coronaviral, or SARS-Cov-2 infection and a bacterial infection, e.g., pneumonia. In certain embodiments, this disclosure contemplates administering an extract or compounds contained therein, in combination with an anti-bacterial agent such as selected from the group comprising of sulfonamides, diaminopyrimidines, quinolones, beta-lactam antibiotics, cephalosporins, tetracyclines, nitrobenzene derivatives, aminoglycosides, macrolide antibiotics, polypeptide antibiotics, nitrofuran derivatives, nitroimidazoles, nicotinic acid derivatives, polyene antibiotics, imidazole derivatives or glycopeptide, cyclic lipopeptides, glycylcyclines and oxazolidinones. In further embodiments, these antibiotics include but are not limited to sulphadiazine, sulfones—[dapsone (DDS) and para-aminosalicylic (PAS)], sulfanilamide, sulfamethizole, sulfamethoxazole, sulfapyridine, trimethoprim, pyrimethamine, nalidixic acids, norfloxacin, ciprofloxacin, cinoxacin, enoxacin, gatifloxacin, gemifloxacin, grepafloxacin, levofloxacin, lomefloxacin, moxifloxacin, ofloxacin, pefloxacin, sparfloxacin, trovafloxacin, penicillins (amoxicillin, ampicillin, azlocillin, carbenicillin, cloxacillin, dicloxacillin, flucloxacillin, hetacillin, oxacillin, mezlocillin, penicillin G, penicillin V, piperacillin), cephalosporins (cefacetrile, cefadroxil, cefalexin, cephaloglycin, cefalonium, cefaloridine, cefalotin, cefapirin, cefatrizine, cefazaflur, cefazedone, cefazolin, cefradine, cefroxadine, ceftezole, cefaclor, cefonicid, ceforanide, cefprozil, cefuroxime, cefuzonam, cefmetazole, cefotetan, cefoxitin, cefcapene, cefdaloxime, cefdinir, cefditoren, cefetamet, cefixime, cefmenoxime, cefodizime, cefoperazone, cefotaxime, cefotiam, cefpimizole, cefpiramide, cefpodoxime, cefteram, ceftibuten, ceftiofur, ceftizoxime, ceftriaxone, cefoperazone, ceftazidime, cefepime), moxalactam, carbapenems (imipenem, ertapenem, meropenem) monobactams (aztreonam), oxytetracycline, chlortetracycline, clomocycline, demeclocycline, tetracycline, doxycycline, lymecycline, meclocycline, methacycline, minocycline, rolitetracycline, chloramphenicol, amikacin, gentamicin, framycetin, kanamycin, neomycin, netilmicin, streptomycin, tobramycin, azithromycin, clarithromycin, dirithromycin, erythromycin, roxithromycin, telithromycin, colistin, bacitracin, tyrothricin, nitrofurantoin, furazolidone, metronidazole, tinidazole, isoniazid, pyrazinamide, ethionamide, nystatin, amphotericin-B, hamycin, miconazole, clotrimazole, ketoconazole, fluconazole, rifampicin, lincomycin, clindamycin, spectinomycin, fosfomycin, loracarbef, polymyxin B, polymyxin B sulfate, procaine, ramoplanin, teicoplanin, vancomycin, and/or nitrofurantoin.

In certain embodiments, an extract or compound disclosed herein can be administered in combination with an anti-inflammatory agent such as alclofenac, alclometasone dipropionate, alpha amylase, amcinafal, amfenac sodium, anakinra, anirolac, balsalazide disodium, bendazac, benoxaprofen, bromelains, broperamole, budesonide, carprofen, cliprofen, clobetasol propionate, clobetasone butyrate, clopirac, cortodoxone, decanoate, deflazacort, depo-testosterone, desonide, desoximetasone, dexamethasone dipropionate, diclofenac potassium, diclofenac sodium, diflorasone diacetate, diflumidone sodium, diflunisal, difluprednate, dimethyl sulfoxide, enolicam sodium, ctodolac, felbinac, fenamole, fenbufen, fenclofenac, fendosal, fenpipalone, fentiazac, flazalone, flufenamic acid, flunisolide acetate, flunixin, flunixin meglumine, fluorometholone acetate, flurbiprofen, fluticasone propionate, furaprofen, halcinonide, halobetasol propionate, ibuprofen, ibuprofen aluminum, ibuprofen piconol, indomethacin, indomethacin sodium, indoprofen, isoxepac, isoxicam, ketoprofen, lofemizole hydrochloride, loteprednol etabonate, meclofenamate sodium, meclofenamic acid, mefenamic acid, mesalamine, methenolone, methenolone acetate, nabumetone, nandrolone, naproxen, naproxen sodium, naproxol, olsalazine sodium, oxaprozin, oxyphenbutazone, oxymetholone, pirfenidone, piroxicam, piroxicam cinnamate, piroxicam olamine, pirprofen, proquazone, proxazole, proxazole citrate, salsalate, stanozolol, sudoxicam, sulindac, suprofen, talniflumate, tenidap, tenidap sodium, tenoxicam, testosterone, testosterone blends, tiopinac, tixocortol pivalate, tolmetin, tolmetin sodium, triclonide, triflumidate, zidometacin, and zomepirac sodium.

In certain embodiments, this disclosure contemplates compositions for use in managing, treating, or preventing coronaviral or other viral infection as disclosed herein comprising materials obtained from partitioning and/or grinding up a plant or section or plant disclosed herein providing a container with processed plant material in a solid or liquid form as a nutritional supplement. The processed plant material may be utilized directly or to extract components of the plant into a suitable solvent for consumption, e.g., particulates, aqueous solution, or tea. In certain embodiments, this disclosure contemplates processed plant material in a container, e.g., box, vial, bottle, capsule, blister pack, jar, drum, bin, burlap, thick paper, or plastic sack, etc., optionally with instruction for manipulating or extracting materials and compounds within the plant material as reported herein for use in managing, treating, or preventing coronaviral or other viral infection as disclosed herein.

### Pharmaceutical Compositions

Pharmaceutical compositions typically comprise an effective amount of an extract or compounds and a suitable pharmaceutical acceptable excipient or carrier. The preparations can be prepared in a manner known per se, which usually involves mixing the compounds according to the disclosure with the one or more pharmaceutically acceptable carriers, and, if desired, in combination with other pharmaceutical active compounds, when necessary, under aseptic conditions. Reference is made to U.S. Pat. Nos. 6,372,778, 6,369,086, 6,369,087 and 6,372,733 and the further references mentioned above, as well as to the standard handbooks, such as the latest edition of Remington's Pharmaceutical Sciences. Pharmaceutically acceptable salts, solvates, and hydrates of the compounds listed are also useful in the method of the disclosure and in pharmaceutical compositions of the disclosure.

In certain embodiments, this disclosure contemplates pharmaceutical compositions comprising extracts and components contained therein for use in methods of treating viral or other microbial infections.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents solvents or vehicles include water, ethanol, polyols (propylene glycol, polyethylene glycol, glycerol, and the like), suitable mixtures thereof, vegetable (such as olive oil, sesame oil) and injectable organic esters such as ethyl oleate.

These compositions may also contain preserving, emulsifying, and dispensing agents. Prevention of the action of microorganisms may be controlled by addition of any of various antibacterial and antifungal agents, example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

In certain embodiments, this disclosure contemplates a pharmaceutical composition in the form of a spray or inhalant, paste, solid, patch, wound dressing, or skin patch. In certain embodiments, this disclosure contemplates a pharmaceutical composition in the form of an ointment or balm.

In certain embodiments, this disclosure contemplates an intravenous formulation with pH buffering agents and tonicity in a range representing physiological values (pH 7 to 8) or for bolus administration, e.g., containing normal saline or dextrose optionally containing pH buffering agents. In certain embodiments, the pharmaceutical composition is in the form of a sterilized pH buffered aqueous salt solution or a saline phosphate buffer between a pH of 6 to 8, optionally comprising a saccharide or polysaccharide.

The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono-or diglycerides, and fatty acids, including oleic acid.

Suitably, the pharmaceutical composition of the disclosure comprises a carrier and/or diluent appropriate for its delivering by injection to a human or animal organism. Such carrier and/or diluent is non-toxic at the dosage and concentration employed. It is selected from those usually employed to formulate compositions for parental administration in either unit dosage or multi-dose form or for direct infusion by continuous or periodic infusion. It is typically isotonic, hypotonic or weakly hypertonic and has a relatively low ionic strength, such as provided by sugars, polyalcohols and isotonic saline solutions. Representative examples include sterile water, physiological saline (e.g., sodium chloride), bacteriostatic water, Ringer's solution, glucose or saccharose solutions, Hank's solution, and other aqueous physiologically balanced salt solutions. The pH of the composition of the disclosure is suitably adjusted and buffered in order to be appropriate for use in humans or animals, typically at a physiological or slightly basic pH (between about pH 8 to about pH 9, with a special preference for pH 8.5). Suitable buffers include phosphate buffer (e.g., PBS), bicarbonate buffer and/or Tris buffer. A typical composition is formulated in 1M saccharose, 150 mM NaCl, 1 mM MgCl₂, 54 mg/L Tween 80, 10 mM Tris pH 8.5. Another typical composition is formulated in 10 mg/mL mannitol, 1 mg/mL HSA, 20 mM Tris, pH 7.2,and 150 mM NaCl.

In certain embodiments, the pharmaceutical composition is in the form of a tablet, pill, capsule, powders, granules, gel, gel capsule, or cream. In such solid dosage forms, the active compound is admixed with at least one inert customary excipient (or carrier) such as sodium citrate or dicalcium phosphate or: (a) fillers or extenders, as for example, starches, lactose, sucrose, glucose, mannitol and silicic acid, (b) binders, as for example, carboxymethylcellulose, alginates, gelatin, polyvinylpyrrolidone, sucrose, and acacia gum, (c) humectants, as for example, glycerol (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof.

Solid dosage forms can be prepared with coatings and shells, such as enteric coatings and others well known in the art. They may contain opacifying agents and can also be of such composition that they release the active compound or compounds in a certain part of the intestinal tract in a delayed manner. In certain embodiments, pharmaceutical composition is in solid form surrounded by an enteric coating. In certain embodiments, the enteric coating comprises methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, or combinations thereof.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like.

The pharmaceutical compositions of the present disclosure can be administered to subjects either topically to the skin, orally, rectally, parenterally (intravenously, intramuscularly, or subcutaneously), intracisternally, intravaginally, intraperitoneally, intravesically, locally (powders, ointments, or drops), or as a buccal or nasal spray.

In certain embodiments, the pharmaceutical compositions are in a form for inhalation. In certain embodiments, the pharmaceutical composition comprises a compound disclosed herein and a propellant. In certain embodiments, an aerosolizing propellant is compressed air, ethanol, nitrogen, carbon dioxide, nitrous oxide, hydrofluoroalkanes (HFAs), or combinations thereof.

In certain embodiments, the disclosure contemplates a pressurized or unpressurized container comprising a compound herein. In certain embodiments, the container is a manual pump spray, inhaler, meter-dosed inhaler, dry powder inhaler, nebulizer, vibrating mesh nebulizer, jet nebulizer, or ultrasonic wave nebulizer.

In certain embodiments, this disclosure relates to pharmaceutical compositions comprising a plant extract, or compound, or mixture of compounds derived thereof as disclosed herein and a pharmaceutically acceptable excipient.

In certain embodiments, the pharmaceutical composition is in the form of a lotion, gel, cream, pill, tablet, capsule, or gel capsule.

In certain embodiments, the pharmaceutical composition is in the form of a spray or inhalant (for nasal spray, throat spray, or inhaled aerosol drug for the lungs), paste, solid, patch, wound dressing, and skin patch.

In certain embodiments, the pharmaceutical composition is in the form of an ointment, including for nasal application such as a Chapstick™ type (balm) application to the lips or nose.

In certain embodiments, the pharmaceutical composition is in the form of a sterilized pH buffered aqueous salt solution or a saline phosphate buffer between a pH of 6 to 8, optionally comprising a saccharide or polysaccharide.

In certain embodiments, the pharmaceutically acceptable excipient is lactose, sucrose, mannitol, triethyl citrate, dextrose, cellulose, methyl cellulose, ethyl cellulose, hydroxyl propyl cellulose, hydroxypropyl methylcellulose, carboxymethylcellulose, croscarmellose sodium, polyvinyl N-pyrrolidone, crospovidone, ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, or acacia gum.

In certain embodiments, the pharmaceutically acceptable excipient is bees wax or plant oils (e.g., almond oil, coconut oil, olive oil, grapeseed oil, cocoa butter, shea butter) and vitamin E to be in line with a topical type e.g., Chapstick™ lip balm, or balm shaped for application to the nose.

In certain embodiments, the pharmaceutically acceptable excipient is an aerosol excipient for administration through a nose or throat spray, or an inhalant.

In certain embodiments, the pharmaceutically acceptable excipient is an aerosolizing propellant such as compressed air, ethanol, nitrogen, carbon dioxide, nitrous oxide, or combinations thereof.

In certain embodiments, the disclosure relates to a liquid or gel formulation optionally further comprising an antibacterial agent, a topical steroid, an anti-inflammatory agent, a promoter of skin barrier function, a skin moisturizer, or combinations thereof. In certain embodiments the antibacterial agent is daptomycin, linezolid, vancomycin, nafcillin, cefazolin, dicloxacillin, clindamycin, rifampin, sulfamethoxazole-trimethoprim, or botanical antibacterial agents, e.g., Melaleuca alternifolia (tea tree oil).

In certain embodiments, the disclosure relates to a solid or liquid soap or lotion comprising an extract or one or more compounds in an extract disclosed herein and a fatty acid.

In certain embodiments, the disclosure relates to a medical device comprising a coating comprising an extract or one or more compounds in an extract disclosed herein.

In certain embodiments, the disclosure relates to a face mask comprising an extract or one or more compounds in an extract disclosed herein embedded in the face mask or a face mask filter.

In certain embodiments, the disclosure relates to a tampon or tampon fibers comprising an extract or one or more compounds in an extract disclosed herein and an absorbent material.

In certain embodiments, the disclosure relates to a wound dressings or wound rinse comprising an extract or one or more compounds in an extract disclosed herein wherein the wound dressing comprises an absorbent pad and optionally an adhesive.

In certain embodiments, the disclosure relates to disinfectant sprays or wipes formulation for surfaces and fomites, comprising an extract and one or one or more compounds in an extract disclosed herein wherein the spray or wipe comprises an extract or one or more compounds in an extract disclosed herein such as a formula including chlorine-based disinfectants.

In certain embodiments, the composition comprising an extract or one or more compounds in an extract disclosed herein is formulated with a compound or compounds which neutralize stomach acid. Alternatively, the pharmaceutical composition containing an extract or one or more compounds in an extract disclosed herein is administered either concurrent with or subsequent to administration of a pharmaceutical composition which neutralize stomach acid. Compounds, such as antacids, which are useful for neutralizing stomach acid include, but are not limited to, aluminum carbonate, aluminum hydroxide, bismuth subnitrate, bismuth subsalicylate, calcium carbonate, dihydroxyaluminum sodium carbonate, magaldrate, magnesium carbonate, magnesium hydroxide, magnesium oxide, and mixtures thereof.

In certain embodiments, the composition comprising an extract or one or more compounds in an extract disclosed herein is formulated with a compound or compounds which inhibit the secretion of stomach acid. Alternatively, the pharmaceutical composition is administered either concurrent with or after administration of a pharmaceutical composition active to inhibit the secretion of stomach acid. Compounds which are useful for inhibiting the secretion of stomach acid include, but are not limited to, ranitidine, nizatidine, famotidine, cimetidine, and misoprostol.

In certain embodiments, the pharmaceutical composition is a directly compressible composition comprising an extract or one or more compounds in an extract disclosed herein and optionally no excipients, additives, or vehicles; however, the formulation may contain a lubricant, such as but not limited to, magnesium stearate. Preferably, the directly compressed formulation is formulated as a tablet of pharmaceutically acceptable hardness (greater than 6 kp, preferably 8-14 kp, and more preferably 10-13 kp).

In certain embodiment, this disclosure contemplates that compounds disclosed herein are used in a substantially purified form. For example, prior to addition to a pharmaceutical formulation the compounds may be purified to contain less than 50%, 40%, 30%, 20%, 10%, or 5%, by weight impurities or derivatives of the compound.

The pharmaceutical preparations of the disclosure are preferably in kit, or in a unit dosage form, and can be suitably packaged, for example in a box, blister, vial, bottle, sachet, ampoule or in any other suitable single-dose or multi-dose holder or container (which can be properly labeled); optionally with one or more leaflets containing product information and/or instructions for use. Generally, such unit dosages will contain between 1 and 1000 mg, and usually between 5 and 500 mg, of the at least one compound of the disclosure e.g., about 10, 25, 50, 100, 200, 300 or 400 mg per unit dosage.

### Identification of Botanical Viral Entry Inhibitors for SARS-COV-2

The Quave Natural Product Library (QNPL) is a collection of over several thousand botanical and fungal extracts and includes many natural supplements. These extracts were tested in a SARS-COV-2 pseudotyped virus system to identify extracts that could inhibit viral entry, specifically the virus spike protein binding to host cells ACE2 receptors. Mammalian cell cytotoxicity assays were run in parallel. Certain extracts reported herein were identified as capable of exhibiting greater than 50% inhibition activity in the wild-type spike pseudotyped model at 20 μg/mL. Some of the extracts exhibited greater than 85% inhibition activity and less than 15% cytotoxicity in the wild-type model. Once these extracts were identified, an interesting pattern emerged indicating many hits were from species that are known to be cardiotoxic due to rich composition of cardiac glycosides. For further selection and testing was done.

TABLE 1
EC50 values were calculated by modeled dose-response
curves against the wild type SARS-CoV-2 pseudovirion
model using non-linear regression.

| | | | | EC₅₀ |
|---|---|---|---|---|
| Extract | | Plant | Extraction | % Inhibition |
| Number | Plant Name | Part | Method | (μg/mL) |
| DMSO | — | — | — | >1.25% v/v |
| Water | — | — | — | >1.25% v/v |
| Hydroxy | — | — | — | 1.14 |
| cholorquine | | | | |
| 220 | Rubus ulmifolius | Roots | EtOH | 22.13 |
| | Schott, Rosaceae | | 2 × 72 hour | |
| | | | maceration | |
| 921 | Vaccinium | Woody | MeOH | 16.61 |
| | tenellum Aiton, | Stem | 2 × 72 hour | |
| | Ericaceae | | maceration | |
| 1104 | Vaccinium | Roots | MeOH | 5.22 |
| | myrsinites Lam., | | 2 × 72 hour | |
| | Ericaceae | | maceration | |
| 1428 | Solidago | Flowers | dH₂O | 2.60 |
| | altissima | | 20 minutes | |
| | L., Asteraceae | | decoction | |
| 1749 | Salix nigra | Bark | 80% EtOH | 7.98 |
| | Marshall, | | (aq) | |
| | Salicaceae | | 2 × 72 hour | |
| | | | maceration | |
| 1783 | Salix nigra | Roots | 80% EtOH | 4.85 |
| | Marshall, | | (aq) | |
| | Salicaceae | | 2 × 72 hour | |
| | | | maceration | |
| 1804 | Pteridium | Rhizomes | 80% EtOH | 7.43 |
| | aquilinum (L.) | | (aq) | |
| | Kuhn, | | 2 × 72 hour | |
| | Dennstaedtiaceae | | maceration | |
| 1886 | Amorpha | Fruit | 80% EtOH | 4.59 |
| | fruticosa | | (aq) | |
| | L., Fabacaeae | | 2 × 72 hour | |
| | | | maceration | |

TABLE 2
EC₅₀ values were calculated by modeled dose-response
curves against the wild-type SARS-CoV-2 pseudovirion
model using non-linear regression.

| | | | | EC₅₀ |
|---|---|---|---|---|
| | | | | % Inhibition |
| Extract | | Plant | Extraction | (μg/mL) ± SEM |
| Number | Plant Name | Part | Method | HEK293-ACE2 |
| 1428 | Solidago | Flowers | dH₂O | 2.60 ± 0.23 |
| | altissima L. | | 20 minutes | |
| | | | decoction | |
| 1749 | Salix nigra | Bark | 80% EtOH (aq) | 7.98 ± 1.32 |
| | Marshall | | 2 × 72 hour | |
| | | | maceration | |
| 1804 | Pteridium | Rhizomes | 80% EtOH (aq) | 7.43 ± 1.5 |
| | aquilinum (L.) | | 2 × 72 hour | |
| | | | maceration | |

These extracts were further validated in a concentration-response assay in a pseudotyped virus model. The EC₅₀ values of the top 3 extracts were all under 10 μg/mL. These 3 extracts (1428, 1749, 180) all exhibited activity (greater than 85% inhibition activity) in the wildtype and variant pseudotyped models. Testing in live SARS-COV-2 confirmed antiviral activity from extracts, Plant A (1428) flowers and Plant B (1804) rhizomes. Further chemical characterization of the major metabolites of these two hits was performed using MS/MS fragmentation data compared with the literature, in silico prediction, and web-based databases.

The results revealed phenylpropanoids, flavonoids, triterpenes, glycosidic terpenes, and fatty acids as the major chemical classes. The next steps of this study seek to identify and isolate purified bioactive compounds to further understand their role in SARS-COV-2 inhibition.

SARS-COV-2 is coronavirus with a crown-like structure created by surface spike proteins. SARS-COV-2 binds to ACE2 receptors of host cells initiating viral entry into ACE2 expressing cells. The extract library was screened against a pseudotyped virus expressing SARS-COV-2 spike protein and encoding firefly luciferase which is capable of infecting human ACE2 expressing HEK293 cells. Once inside the cells resulting firefly luciferase transcription and translations can be monitored by fluorescence detection. Library extracts capable of inhibiting viral entry reduce or eliminate fluorescence detection indicating an ability to interfere with spike protein and ACE2 protein interactions.

TABLE 3
shows data confirming the antiviral activity
of extract 18014 by virus yield reduction.

| | Anti-SARS-CoV2 | | Anti-SARS-CoV2 | | Anti-SARS-CoV2 | | Cytotoxicity | |
|---|---|---|---|---|---|---|---|---|
| | (Vero) | | (Calu3) | | (Caco2) | | (CC50) | |
| Extract | EC50 | EC90 | EC50 | EC90 | EC50 | EC90 | PBM | Vero |
| 1804 | 4.7 | 9.5 | 28.9 | >20 | 42.5 | >20 | 80.8 | 88.1 |
| (μg/mL) | | | | | | | | |
| 1428 | 4.5 | 9.9 | 20.0 | >20 | 27.2 | >20 | >100 | >100 |
| (μg/mL) | | | | | | | | |
| Remdesivir | 0.6 | 1.6 | 0.002 | 0.02 | 0.1 | 0.4 | 1.2 | 60.3 |
| (μM) | | | | | | | | |

Numbers in parentheses indicate percent inhibition at the highest concentration tested. Cytotoxicity assays measuring the cell proliferation in PBM and Vero cells were performed in parallel to the antiviral assays.

A phylogenetic assessment of the 185 plant species demonstrating bioactivity (greater than 50% inhibition) identified predominant activity in the asterid families and many species in Fagales and Rosales within the fabid clade of plants. Certain botanicals are useful for therapies to combat COVID-19. Chemical analysis of top two candidates was performed using high resolution mass spectrometry. The major metabolites from both extracts were tentatively identified based on the MS/MS fragmentation data compared with literature, in silico prediction, and web-based databases. In extract 1804 (from Pteridium aquilinum), a variety of metabolites were detected, including proanthocyanidins including dimeric procyanidins, trimeric procyanidin, prodelphinidin phenylpropanoids, flavonoids, and triterpenes. In extract 1428 (from Solidago altissima), phenylpropanoids, glycosidic triterpenoids, and fatty acids were detected as major chemical classes.

In certain embodiments, this disclosure contemplates using extracts and/or chemicals disclosed herein for therapeutic uses in treating or preventing coronavirus or other viral infections. In certain embodiments, the plant is in the Solidago genus. In certain embodiments, the plant is Solidago altissima (Extract 1428). In certain embodiments, the segment is the flower. In certain embodiments, the segment is the flower, root, rhizomes, bark, fruit, leaf, stem, or seed. In certain embodiments, the extract is obtained from using a solvent selected from water, room temperature water, below room temperature water, boiling water, MeOH and EtOH, and aqueous MeOH, aqueous EtOH. In certain embodiments, it is contemplated that the extract is obtained using Soxhlet extractor, decoction, maceration, microwave-assisted extraction, supercritical carbon dioxide extraction, or with assistance of sonication. In certain embodiments, the extract comprises a compound such as a quinic acid derivative, coumaroylquinic acid, 3-O-feruloylquinic acid, a glycosidic triterpene derivative, fatty acid, trihydroxy octadecenoic acid, ester, derivative, or combinations thereof.

In certain embodiments, this disclosure contemplates using extracts and/or chemicals disclosed herein for therapeutic uses in treating or preventing coronavirus or other viral infections. In certain embodiments, the plant is in the Pteridium genus. In certain embodiments, the plant is Pteridium aquilinum (Extract 1804). In certain embodiments, the plant segment is the rhizomes. In certain embodiments, the segment is the flower, root, rhizomes, bark, fruit, leaf, stem, or seed. In certain embodiments, the solvent is selected from water, room temperature water, below room temperature water, boiling water, MeOH and EtOH, and aqueous MeOH, aqueous EtOH. In certain embodiments, it is contemplated that the extract is obtained using Soxhlet extractor, decoction, maceration, microwave-assisted extraction, supercritical carbon dioxide extraction, or with assistance of sonication. In certain embodiments, the extract comprises a compound such as a glycosidic phenolic compound, benzoyl hexose, caffeoyl hexose, a glycosidic phenolic compound, procyanidin derivatives, A-type procyanidin dimer and/or prodelphinidin (one double linkage between first and second units), A-type procyanidin trimer (one double linkage between first and second units), quinic acid derivatives such as coumaroylquinic acid, caffeoylquinic acid, cholestane-type sterol, ester, ester derivative, or combinations thereof.

In certain embodiments, this disclosure contemplates using extracts and/or chemicals disclosed herein for therapeutic uses in treating or preventing coronavirus or other viral infections. In certain embodiments, the plant is in the Salix genus. In certain embodiments, the plant is Salix nigra (Extract. 1749). In certain embodiments, the segment is the bark. In certain embodiments, the segment is the flower, root, rhizomes, bark, fruit, leaf, stem, or seed. In certain embodiments, the solvent is selected from water, room temperature water, below room temperature water, boiling water, MeOH and EtOH, and aqueous MeOH, aqueous EtOH. In certain embodiments, it is contemplated that the extract is obtained using Soxhlet extractor, decoction, maceration, microwave-assisted extraction, supercritical carbon dioxide extraction, or with assistance of sonication.

In certain embodiments, the plant is Salix nigra (Extract. 1783). In certain embodiments, the segment is the root. In certain embodiments, the segment is the flower, root, rhizomes, bark, fruit, leaf, stem, or seed. In certain embodiments, the solvent is selected from water, room temperature water, below room temperature water, boiling water, MeOH and EtOH, and aqueous MeOH, aqueous EtOH. In certain embodiments, it is contemplated that the extract is obtained using Soxhlet extractor, decoction, maceration, microwave-assisted extraction, supercritical carbon dioxide extraction, or with assistance of sonication.

In certain embodiments, this disclosure contemplates using extracts and/or chemicals disclosed herein for therapeutic uses in treating or preventing coronavirus or other viral infections. In certain embodiments, the plant is in the Rubus genus. In certain embodiments, the plant is Rubus ulmifolius (Extract. 220). In certain embodiments, the segment is the root. In certain embodiments, the segment is the flower, root, rhizomes, bark, fruit, leaf, stem, or seed. In certain embodiments, the solvent is selected from water, room temperature water, below room temperature water, boiling water, MeOH and EtOH, and aqueous MeOH, aqueous EtOH. In certain embodiments, it is contemplated that the extract is obtained using Soxhlet extractor, decoction, maceration, microwave-assisted extraction, supercritical carbon dioxide extraction, or with assistance of sonication.

In certain embodiments, this disclosure contemplates using extracts and/or chemicals disclosed herein for therapeutic uses in treating or preventing coronavirus or other viral infections. In certain embodiments, the plant is in the Vaccinium genus. In certain embodiments, the plant is Vaccinium tenellum (Extract. 921). In certain embodiments, the segment is the stem. In certain embodiments, the segment is the flower, root, rhizomes, bark, fruit, leaf, stem, or seed. In certain embodiments, the solvent is selected from water, room temperature water, below room temperature water, boiling water, MeOH and EtOH, and aqueous MeOH, aqueous EtOH. In certain embodiments, it is contemplated that the extract is obtained using Soxhlet extractor, decoction, maceration, microwave-assisted extraction, supercritical carbon dioxide extraction, or with assistance of sonication.

In certain embodiments, the plant is Vaccinium myrsinites (Extract. 1104). In certain embodiments, the segment is the root. In certain embodiments, the segment is the flower, root, rhizomes, bark, fruit, leaf, stem, or seed. In certain embodiments, the solvent is selected from water, room temperature water, below room temperature water, boiling water, MeOH and EtOH, and aqueous MeOH, aqueous EtOH. In certain embodiments, it is contemplated that the extract is obtained using Soxhlet extractor, decoction, maceration, microwave-assisted extraction, supercritical carbon dioxide extraction, or with assistance of sonication.

In certain embodiments, this disclosure contemplates using extracts and/or chemicals disclosed herein for therapeutic uses in treating or preventing coronavirus or other viral infections. In certain embodiments, the plant is in the Amorpha genus. In certain embodiments, the plant is Amorpha fruticosa (Extract. 1886). In certain embodiments, the segment is the fruit. In certain embodiments, the segment is the flower, root, rhizomes, bark, fruit, leaf, stem, or seed. In certain embodiments, the solvent is selected from water, room temperature water, below room temperature water, boiling water, MeOH and EtOH, and aqueous MeOH, aqueous EtOH. In certain embodiments, it is contemplated that the extract is obtained using Soxhlet extractor, decoction, maceration, microwave-assisted extraction, supercritical carbon dioxide extraction, or with assistance of sonication.

### Chemical Analysis of Extracts (1804 and 1428)

Chemical analysis of two extracts were performed using high-resolution mass spectrometry. The major metabolites from both extracts were tentatively identified based on the MS/MS fragmentation data compared with literature, in silico prediction, and web-based databases. In extract 1804 (from rhizomes of Pteridium aquilinum), a variety of metabolites were detected, including phenylpropanoids, proanthocyanidins, flavonoids, and triterpenes. In extract 1428 (from flowers of Solidago altissima), phenylpropanoids, glycosidic triterpenoids, and fatty acids were detected as major chemical classes (Tables 4 and 5).

TABLE 4
Identified major metabolites of 1804 extract.

| peak | | tR | observed m/z | molecular |
|---|---|---|---|---|
| number | identification | (min) | [M − H]− | formula |
| 1 | benzoyl hexose | 1.94 | 329.0875 | C14H18O9 |
| | derivative | | | |
| 2 | caffeoyl hexose | 2.37 | 341.0881 | C15H18O9 |
| 3 | glycosidic | 2.44 | 371.0978 | C16H20O10 |
| | phenolic compound | | | |
| 4 | A-type | 3.27 | 879.1776 | C45H36O19 |
| | procyanidin | | | |
| | dimer + | | | |
| | prodelphinidin | | | |
| | (one double | | | |
| | linkage between | | | |
| | first and second | | | |
| | units) | | | |
| 5 | A-type | 3.66 | 863.1833 | C45H36O18 |
| | procyanidin | | | |
| | trimer (one | | | |
| | double linkage | | | |
| | between first and | | | |
| | second units) | | | |
| 6 | caffeoylquinic | 4.34 | 641.1514 | C31H30O15 |
| | acid derivatives | | | |
| 7 | glycosidic | 4.41 | [M + HCOOH − | C20H28O10 |
| | phenolic | | H]− | |
| | compound | | 473.1670 | |
| 8 | cholestane-type | 6.20 | [M + HCOOH − | C28H46O8 |
| | sterol | | H]− | |
| | | | 509.3145 | |

TABLE 5
Tentatively identified major metabolites of 1428 extract.

| peak | | | observed m/z | molecular |
|---|---|---|---|---|
| number | identification | t_{R} (min) | [M − H]⁻ | formula |
| 1 | coumaroylquinic | 3.56 | 337.0928 | C₂₁H₃₀O₁₂ |
| | acid | | | |
| 2 | 3-O- | 3.88 | 367.1030 | C₁₇H₂₀O₉ |
| | feruloylquinic | | | |
| | acid | | | |
| 3 | coumaroylquinic | 4.02 | 337.0926 | C₁₆H₁₈O₈ |
| | acid | | | |
| 4 | unidentified | 5.94 | 233.0965 | C₁₁H₁₉ClO₃ |
| 5 | glycosidic | 6.34 | 1643.7333 | C₇₅H₁₂₀O₃₉ |
| | triterpene | | | |
| | derivative | | | |
| 6 | glycosidic | 6.52 | 1629.7181 | C₇₄H₁₁₈O₃₉ |
| | triterpene | | | |
| | derivative | | | |
| 7 | trihydroxy | 6.62 | 329.2334 | C₁₈H₃₄O₅ |
| | octadecenoic | | | |
| | acid | | | |

LC-MS analysis. The dried extracts of 1749, and 1428 were dissolved with MeOH/H2O (5:5, v/v) 5 by 10.0 mg/mL concentration for LC-MS analysis. The 1428 extracts (7.8 g) were further subjected to column chromatography on an HP-20™ resin (3×14 cm id) and eluted by water, 50% methanol, methanol, and acetone to yield 4 fractions, and each fraction was dissolved with MeOH/dH2O (5:5, v/v) by 5.0 mg/mL for the LC-MS analysis. The LC-MS analysis was performed on an Agilent 1290 Infinity™ II UHPLC system coupled to an Agilent 6545XT™ 10 QTOFMS, which was equipped with a Dual AJS™ ESI Ion Source. Chromatographic separations were performed on a Zorbax Eclipse™ XDBC18 (100×2.1 mm, 1.8 μM) column coupled with Zorbax Eclipse™ XDB-C18 (5×2.1 mm, 1.8 μM) guard column.

The mobile phase was comprised of H20 (A) and acetonitrile (B), both of which were acidified with 0.1% formic acid. The column temperature and sample organizer were maintained at 40° C. and 15° C., respectively. A stepwise gradient method at a constant flow rate of 0.4 mL/min was used to elute the column with the following conditions: 5-5% B (0.0-0.5 min); 5-25% B (0.5-4.0 min); 25-60% B (4.0-7.0 min); 60-100% B (7.0-9.0 min); and 100-100% B (9.0-10.5 min), followed by a return to the starting conditions at 10.6 min and 1.4 min of reconditioning the column (total runtime of 12.0 min). Analyses of the samples (2.0 μL, injection volume) were performed in the negative ion mode in both profile and centroid mode.

The ESI conditions were set as follows: the capillary voltage was 4.0 kV, the nozzle voltage was 2000 V for negative mode, the fragmentor was 100 V, the drying gas temperature and flow were set to 325° C. and 13 L/min, respectively, and sheath gas temperature and flow were 275° C. and 12 L/min, respectively, and the nebulizer was operating at 35 psi. Nitrogen served both as the nebulizer gas and the dry gas. The Auto-MS/MS mode was used with an MS range of m/z 100-1700 and an MS2 range of m/z 50-1700, at 7 spectra/s and 5 spectra/s, respectively. The narrow isolation (˜1.3 m/z) width was used. The collision energy was set by the formula based on the m/z and charge of the precursor (condition 1: slope of 3.8 and an offset of 20, condition 2: slope of 2.0 and an offset of 6). The maximum precursors per cycle are set to 5, with the absolute precursor threshold set to 500 (relative threshold 0.015%) and active exclusion after 3 scans and released after 0.1 min were performed.

### Infectious Antiviral Activity Assays

To determine the potential antiviral effects of the extracts against in vitro replication of

SARS-COV-2 in cell culture, a confluent monolayer of Vero cells in a 96-well cell culture microplate was treated with 20 μg/mL of compound followed by inoculation with 0.1 MOI of the virus. To assess the antiviral activity, a virus yield reduction assay using specific qRT-PCR for each virus was performed. The half maximal effective concentration (EC₅₀) was calculated.

### Concentration Dependent Antiviral Assay

The antiviral activity of extracts 1428 and 1804 have been further confirmed by virus yield reduction assay using specific qRT-PCR for SARS-COV-2 by measuring the RNA copy number of the virus after 2-days post-treatment (for Vero cells) and after 3-days post-treatment (for Calu-3 and Caco-2 cells) in supernatant of treated-infected cells in a dose response manner (extracts tested at 0.3-20 μg/mL, remdesivir tested at 1-100 μM). One step qRT-PCR was carried out in a final volume of 10 μL containing extracted viral RNA, specific probe/primer mix and qScript-Tough™ master mix. Quantitative PCR measurements were performed using LightCycler® 480 PCR system (Roche, Germany) according to manufacturer's protocol.

What is claimed is:

1. A method of treating a coronavirus infection or other microbial infection comprising administering an effective amount of a plant extract, or compound, or combination of compounds derived therefrom to a subject in need thereof, wherein the plant extract is obtained by a process of contacting a segment of a plant a with a solvent providing an extract, wherein the plant is Solidago altissima (Extract 1428).

2. The method of claim 1, wherein the segment is the flower.

3. The method of claim 3, wherein the solvent is selected from boiling water, MeOH and EtOH, and aqueous MeOH, aqueous EtOH.

4. The method of claim 1 wherein the compound is a quinic acid derivative, coumaroylquinic acid, 3-O-feruloylquinic acid, a glycosidic triterpene derivative, fatty acid, trihydroxy octadecenoic acid, ester, derivative, or combinations thereof.

5. The method of claim 1, wherein the subject is diagnosed with SARS-COV-2, SARS-COV-1, MERS-COV, HCoV-229E, HCoV-OC43, HCoV-NL63, or HCoV-HKU1, an endemic human coronavirus, epidemic coronavirus, or pandemic coronavirus.

6. The method of claim 1, wherein the extract, compound, or compounds, esters or derivatives are administered to the subject with 5 days or after 5 days, or 1 week or 1 month after symptom onset.

7. A pharmaceutical composition comprising a plant extract, or compound, or mixture of compounds derived thereof as in claim 1 and a pharmaceutically acceptable excipient.

8. The pharmaceutical composition of claim 7 in the form of a lotion, gel, cream, pill, tablet, capsule, or gel capsule.

9. The pharmaceutical composition of claim 7 in the form of a spray or inhalant, paste, solid, patch, wound dressing, or skin patch.

10. The pharmaceutical composition of claim 7 in the form of an ointment or balm.

11. The pharmaceutical composition of claim 7 in the form of a sterilized pH buffered aqueous salt solution or a saline phosphate buffer between a pH of 6 to 8, optionally comprising a saccharide or polysaccharide.

12. The pharmaceutical composition of claim 7 wherein the pharmaceutically acceptable excipient is lactose, sucrose, mannitol, triethyl citrate, dextrose, cellulose, methyl cellulose, ethyl cellulose, hydroxyl propyl cellulose, hydroxypropyl methylcellulose, carboxymethylcellulose, croscarmellose sodium, polyvinyl N-pyrrolidone, crospovidone, ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, or acacia gum.

13. The pharmaceutical composition of claim 7 wherein the pharmaceutically acceptable excipient is bees wax, plant oil, almond oil, coconut oil, olive oil, grapeseed oil, cocoa butter, shea butter, or vitamin E.

14. The pharmaceutical composition of claim 7 wherein the pharmaceutically acceptable excipient is aerosol excipient, an inhalant, aerosolizing propellant, compressed air, ethanol, nitrogen, carbon dioxide, or nitrous oxide.

15. A method of treating a coronavirus infection or other microbial infection comprising administering an effective amount of a plant extract, or compound, or combination of compounds derived therefrom to a subject in need thereof, wherein the plant extract is obtained by a process of contacting a segment of a plant a with a solvent providing an extract, wherein the plant is Pteridium aquilinum (Extract 1804).

16. The method of claim 15, wherein the segment is the rhizomes.

17. The method of claim 15, wherein the solvent is selected from boiling water, MeOH and EtOH, and aqueous MeOH, aqueous EtOH.

18. The method of claim 15 wherein the compound is glycosidic phenolic compounds, benzoyl hexose, caffeoyl hexose, a glycosidic phenolic compound, procyanidin derivatives, A-type procyanidin dimer plus prodelphinidin (one double linkage between first and second units), A-type procyanidin trimer (one double linkage between first and second units), cholestane-type sterol, ester, ester derivative, or combinations thereof.

19. The method of claim 15, wherein the subject is diagnosed with SARS-COV-2, SARS-COV-1, MERS-COV, HCOV-229E, HCoV-OC43, HCoV-NL63, or HCoV-HKU1, an endemic human coronavirus, epidemic coronavirus, or pandemic coronavirus 1.

20. A pharmaceutical composition comprising a plant extract, or compound, or mixture of compounds derived thereof as in claim 15 and a pharmaceutically acceptable excipient.
